(19) United States
(12) Patent Application Publication
(10) Pub. No.: US 2022/0359038 A1
(43) Pub. Date: Nov. 10, 2022

(54) Systems and Methods for the Efficient Identification and Extraction of Sequence Paths in Genome Graphs

(71) Applicant: KONINKLIJKE PHILIPS N.V., EINDHOVEN (NL)

(72) Inventor: Yee Him Cheung, Boston, MA (US)

(21) Appl. No.: 17/762,097

(22) PCT Filed: Sep. 29, 2020
(86) PCT No.: PCT/EP2020/077158

§ 371 Date: Mar. 21, 2022

Related U.S. Application Data
(60) Provisional application No. 62/908,627, filed on Oct. 1, 2019.

(51) Int. Cl. G16B 30/00; G16B 40/20; G16B 45/00

(52) U.S. Cl. CPC G16B 30/00; G16B 40/20; G16B 45/00

(57) ABSTRACT

A method for storing, by a processor, a genomic graph representing a plurality of individual genomes, including: storing a linear representation of a reference genome in a data storage; receiving a first genome; identifying variations in the first genome from the reference genome; generating graph edges for each variation in the first genome from the reference genome; generating for each generated graph edge: an edge identifier that uniquely identifies the current edge in the genome graph; a start edge identifier that identifies the edge from which the current edge branches out; a start position that indicates the position on the start edge that serves as an anchoring point for the current edge; an end edge identifier that identifies the edge into which the current edge joins in; an end position that indicates the position on the end edge that serves as an anchoring point for the current edge; and a sequence indicating the nucleotide sequence of the current edge; and storing the edge identifier, start edge identifier, start position, end edge identifier, end edge position, and sequence for each generated graph edge in the data storage. Based on this genome graph data structure, we further propose a scheme for specifying a path, which may traverse one or more edges, and the ways to extend existing genomic data formats such as SAM, VCF and MPEG-G to support the use of genome graph reference using our proposed coordinate system.

### TECHNICAL FIELD

Various exemplary embodiments disclosed herein relate generally to a system and methods for the efficient identification and extraction of sequence paths in genome graphs.

### BACKGROUND

A linear reference genome is currently the most prevalent model used in the processing and analysis, such as read alignment and variant calling, of next generation sequencing (NGS) data. It is based on the use of a single, preferred tiling path to produce a single consensus representation of the genome. For example, the linear reference NCBI GRCh38 (Hg38) is a composite genome with approximately 93% of the primary assembly consisting of sequences from 11 individuals. Despite its popularity among scientists, due to its ease of reference and lower requirement for computational analysis, a single tiling path is insufficient to represent the allelic diversity in complex genomic regions for most mammalian genomes. With a great deal of common genomic variation excluded, it introduces a pervasive reference bias, which has a negative impact on the accuracy of downstream analysis. For instance, if the genomic region that contains a clinically relevant mutation of a patient is significantly different from the reference genome, then the sequencing reads of the patient in that region will not be correctly mapped to the reference, thus resulting in a missed call of the variant that could be critical for diagnosis and treatment. The latest human reference genome GRCh38 tried to improve the representation of allelic diversity by including alternate loci scaffolds in regions with known alternate haplotypes (e.g., major histocompatibility complex (MHC)), high variability (e.g., olfactory receptor) and large structural variants greater than 5 Kb. Many scientists, however, believe that one of the most effective ways to represent the complex genomic diversity in a reference cohort is through the adoption of a genome graph, where genomic variations are captured as edges associated with different nucleotide sequences. With the advancement in bioinformatics algorithms and computational power, it is foreseen that graph-based genome analysis will become one of the mainstream approaches for genomic studies.

### SUMMARY

A summary of various exemplary embodiments is presented below. Some simplifications and omissions may be made in the following summary, which is intended to highlight and introduce some aspects of the various exemplary embodiments, but not to limit the scope of the invention. Detailed descriptions of an exemplary embodiment adequate to allow those of ordinary skill in the art to make and use the inventive concepts will follow in later sections.

Various embodiments relate to a method for storing, by a processor, a genomic graph representing a plurality of individual genomes, including: storing a linear representation of a reference genome in a data storage; receiving a first genome; identifying variations in the first genome from the reference genome; generating graph edges for each variation in the first genome from the reference genome; generating for each generated graph edge: an edge identifier that uniquely identifies the current edge in the genome graph; a start edge identifier that identifies the edge from which the current edge branches out; a start position that indicates the position on the start edge that serves as an anchoring point for the current edge; an end edge identifier that identifies the edge into which the current edge joins in; an end position that indicates the position on the end edge that serves as an anchoring point for the current edge; and a sequence indicating the nucleotide sequence of the current edge; and storing the edge identifier, start edge identifier, start position, end edge identifier, end edge position, and sequence for each generated graph edge in the data storage.

Further various embodiments relate to a non-transitory machine-readable storage medium encoded with instructions for storing, by a processor, a genomic graph representing a plurality of individual genomes, the non-transitory machine-readable storage medium including: instructions for storing a linear representation of a reference genome in a data storage; instructions for receiving a first genome; instructions for instructions for identifying variations in the first genome from the reference genome; instructions for generating graph edges for each variation in the first genome from the reference genome; instructions for generating for each generated graph edge: an edge identifier that uniquely identifies the current edge in the genome graph; a start edge identifier that identifies the edge from which the current edge branches out; a start position that indicates the position on the start edge that serves as an anchoring point for the current edge; an end edge identifier that identifies the edge into which the current edge joins in; an end position that indicates the position on the end edge that serves as an anchoring point for the current edge; and a sequence indicating the nucleotide sequence of the current edge; and instructions for storing the edge identifier, start edge identifier, start position, end edge identifier, end edge position, and sequence for each generated graph edge in the data storage.

Various embodiments are described, further including: generating for each generated graph edge a length indicating the length of the sequence; and storing the length in the data storage.

Various embodiments are described, wherein the edge identifier, start edge identifier, start position, end edge identifier, end edge position, sequence, and length for each generated graph edge are stored as a row in a data table.

Various embodiments are described, wherein the edge identifier, start edge identifier, start position, end edge identifier, end edge position, and sequence for each generated graph edge are stored as a row in a data table.

Various embodiments are described, further including concatenating the sequences for each of the generated edges and storing them in a sequence data structure separate from the data table.

Various embodiments are described, further including specifying a path in the genome graph, wherein the path is defined by: a position indicating the starting point of the path including a chromosome identifier; the edge identifier, and a base position; a path length indicating the total number of nucleotides in the path; and a trail including a cascade of edge identifiers traversed by the path using delimiters.

Various embodiments are described, wherein the delimiters include a branch-out delimiter and an endpoint delimiter.

Various embodiments are described, further including simplifying the trail by removing edge identifiers from the trail based upon assumptions of the default priorities of the next edges.

Various embodiments are described, wherein the edge identifier includes a group identifier and an edge index, wherein the group identifier identifies a group of related edges and the edge index uniquely identifies an edge within a group.

Various embodiments are described, wherein the group identifier identifies a group of edges from a same origin, such as an individual sample.

Various embodiments are described, further including extending a sequencing read alignment file (SAM) by adding the additional data fields: the edge identifier of the specific edge to which the beginning of the read is aligned; an ENEXT identifier of the specific edge to which the next read of the template is primarily aligned; a trail indicating the delimited sequence of edge transitions taken by the path to which the read is aligned; and TNEXT indicating the delimited sequence of edge transitions taken by the path to which the next read of the template is primarily aligned.

Various embodiments are described, further including extending a variant call file (VCF) by adding the additional data fields: a trail indicating sequence of edge transitions that lead to the location of the variant; and a distance indicating the distance in the number of bases from the upstream anchor point to the variant.

Various embodiments are described, further including extending a MPEG-G file by adding the additional data fields: a coordinate_scheme field that indicates to a decoder how the genomic coordinates should be interpreted; and a trail field in each genomic record to indicate for each alignment position in mapping_pos the sequence of edge transitions to which all template segments in the same record are aligned; using the MPEG-G file data fields as follows: the seq_ID indicates an edge in a chromosome to which the beginning of the first read is mapped; the split_seq_ID indicates the beginning edges of any split segment alignments; and the positions mapping_pos and split_pos count from the beginning of the edges respectively in seq_ID and split_seq_ID.

Various embodiments are described, further includingdetermining anchor points on a specified edge by extending a MPEG-G file by running Dijkstra's algorithm beginning from one of the endpoints of specified edge using a number of bases in between connecting nodes as distance until the specified edge is on a spanning tree with a shortest distance among all leaf nodes, except for those that cannot be extended further.

Various embodiments are described, further including determining the sequence along a path in the genome sequence further comprising: traversing a trail defining the path, wherein the trail includes a cascade of edge identifiers traversed by the path; and concatenating the sequences of each of the edges identified along the path.

### BRIEF DESCRIPTION OF THE DRAWINGS

In order to better understand various exemplary embodiments, reference is made to the accompanying drawings, wherein:

FIG. 1 illustrates an example of a partial genome graph for chromosome Chr_N;

FIG. 2 illustrates a first example path;

FIG. 3 illustrates a second example path;

FIG. 4 illustrates a third example path;

FIG. 5 illustrates a fourth example path; and

FIG. 6 illustrates an exemplary hardware diagram for implementing a system for storing and processing genome graphs.

To facilitate understanding, identical reference numerals have been used to designate elements having substantially the same or similar structure and/or substantially the same or similar function.

### DETAILED DESCRIPTION

The description and drawings illustrate the principles of the invention. It will thus be appreciated that those skilled in the art will be able to devise various arrangements that, although not explicitly described or shown herein, embody the principles of the invention and are included within its scope. Furthermore, all examples recited herein are principally intended expressly to be for pedagogical purposes to aid the reader in understanding the principles of the invention and the concepts contributed by the inventor(s) to furthering the art and are to be construed as being without limitation to such specifically recited examples and conditions. Additionally, the term, “or,” as used herein, refers to a non-exclusive or (i.e., and/or), unless otherwise indicated (e.g., “or else” or “or in the alternative”). Also, the various embodiments described herein are not necessarily mutually exclusive, as some embodiments can be combined with one or more other embodiments to form new embodiments.

The linear reference genome is currently the most prevalent model used in the processing and analysis of next generation sequencing (NGS) data. Despite its popularity among scientists, a single tiling path is insufficient to represent the allelic diversity in complex genomic regions for most mammalian genomes. Many scientists believe that the most effective way to represent the complex genomic diversity in a reference cohort is through the adoption of a genome graph, where genomic variations are captured as edges associated with different nucleotide sequences. One major challenge for genome graphs is having a coordinate system to effectively and unambiguously identify sequence loci in a graph. The embodiments described herein propose and describe a data structure and coordinate system for defining a genome graph, and the system and methods for the identification and extraction of sequence paths based on the proposed data structure. Further the potential applications of the proposed coordinate system for extending existing genomic data file formats are described. Such file formats include sequencing read alignment file (SAM), variant call file (VCF), and MPEG-G (the international standard for the compression of sequencing reads) to support the location of reads and variants mapped to a genome graph reference.

One major challenge for a genome graph is having a coordinate system for effectively and unambiguously identifying sequence loci in a graph. The Computational Pan-Genomics Consortium has identified some desirable properties of the linear model that should be preserved in the graph model. These properties include: monotonicity which includes increasing coordinates of successive bases in 5′ to 3′ direction; readability where the data is compact and human interpretable; and spatiality which includes physically close bases should have similar coordinates.

The genome graph data structure will now be described. A genome graph may be built by starting from a linear reference genome and then progressively expanding it by adding more samples, each introducing a group of new edges (i.e., genomic variations) to the existing graph through read alignment and variant detection. A data structure is proposed for the efficient representation of a genome graph with the following key elements:
(i) Chromosome ID—an identifier for the specific chromosome.
(ii) Group ID—the ID of a group of edges, usually originated from the same sample.
(iii) Edge Index—an increasing number that uniquely identifies an edge within a group. It may be assigned in such a way that an edge at a downstream position is coupled with a larger index, so that it is indicative of the relative position of the edges.
(iv) Edge ID—an ID that uniquely identifies an edge in a chromosome, e.g., by the concatenation of Group ID and Edge Index.
(v) Edge Sequence—a string of characters that defines the sequence of nucleotides in an edge. In the case of deletion, it may be an empty string.

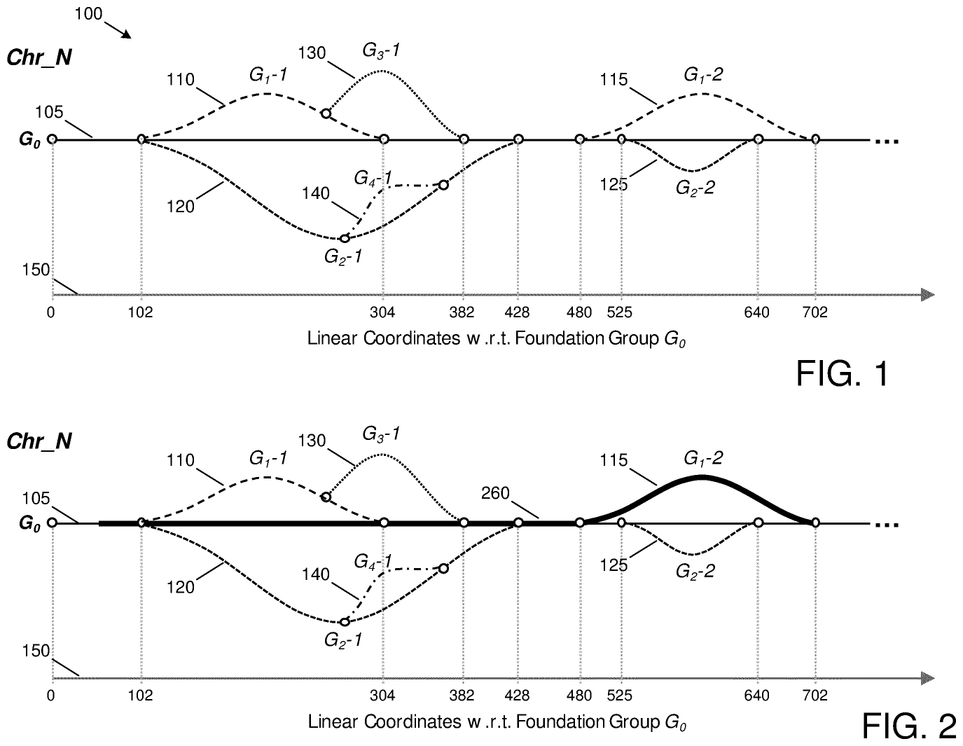

FIG. 1 illustrates an example of a partial genome graph for chromosome Chr_N. The graph 100 includes five groups, G₀, G₁, G₂, G₃, and G₄, shown using different lines and edges labelled by their Edge IDs. The connecting points or nodes are marked by dots. G₀ 105 represents the foundation group that includes a linear sequence stretching from the beginning to the end of the chromosome and serving as the backbone of the graph 100. Technically, each segment on the backbone sequence in between two nodes is an edge in G₀ 105. However, due to its linear nature, an edge index for G₀ 105 is redundant and may be ignored. In this specific case for G₀ 105, the Edge ID is simply the Group ID. For compatibility with linear models, standard genome assembly, such as human NCBI GRCh38 (Hg38), may be used to define the backbone sequence. The incorporation of a backbone sequence is however optional. Although not indicated in the graph, all edges are directional from left to right (5′ to 3′). For example, the Edge ID G₁-1 110 refers to Edge 1 in Group G₁. The graph 100 shows a linear sequence stretching from the beginning to the end of the chromosome with each base marked by a coordinate value 150. Note that the graph is not drawn to scale of the number of nucleotide bases.

The graph 100 includes five groups G₀, G₁, G₂, G₃, and G₄. As described above G₀ 105 is the foundation group. The group G₁ includes two edges G₁-1 110 and G₁-2 115 that illustrate variations from the foundation group G₀ 105. The group G₂ includes two edges G₂-1 120 and G₂-2 125 that illustrate variations from the foundation group G₀ 105. The group G₃ includes an edge G₃-1 130 that illustrates a variation from the edge G₁-1 110 and the foundation group G₀ 105. The group G₄ includes an edge G₄-1 140 that illustrates a variation from the edge G₂-1 120.

Note that to avoid any ambiguity in path definition, an edge cannot join in and then branch out at the same position on another edge. If this happens, it should be split into multiple edges at those junction points.

Using the aforementioned elements, the genome graph of a chromosome can then be stored in a data matrix with the following fields:
(i) EdgeID—an ID that uniquely identifies an edge in the graph genome and serves as a row key to the matrix.
(ii) StartEdge—the ID of the edge from which the current edge branches out.
(iii) StartPos—the position on StartEdge that serves as the 5′ (or left) anchoring point for the current edge. It could refer to the position of the base before which the edge branches out.
(iv) EndEdge—the ID of the edge to which the current edge joins in.
(v) EndPos—the position on EndEdge that serves as the 3′ (or right) anchoring point for the current edge. It could refer to the position of the base before which the edge joins in.
(vi) Seq—the string of the nucleotide sequence of the current edge, which may be an empty string for deletions.
(vii) Length (Optional)—derived from the length of the nucleotide sequence in Seq.

An example of the data structure for the partial genome graph in FIG. 1 is shown in Table 1.

TABLE 1

| EdgeID | StartEdge | StartPos | EndEdge | EndPos | Seq | Length |
|---|---|---|---|---|---|---|
| G₁-1 | G₀ | 102 | G₀ | 304 | ATTCA . . . | 284 |
| G₁-2 | G₀ | 480 | G₀ | 702 | GTCAA . . . | 292 |
| G₂-1 | G₀ | 102 | G₀ | 428 | ATATA . . . | 420 |
| G₂-2 | G₀ | 525 | G₀ | 640 | CCGGC . . . | 151 |
| G₃-1 | G₁-1 | 204 | G₀ | 382 | CAAGG . . . | 255 |
| G₄-1 | G₂-1 | 200 | G₂-1 | 302 | GAATT . . . | 112 |
| . . . | . . . | . . . | . . . | . . . | . . . | . . . |

The data structure for the example genome graph in FIG. 1 has a separately defined backbone sequence for G₀ 105 and is assumed to provide the basis for Table 1. Each row in Table 1 corresponds to an edge in the graph identified by an edge identifier EdgeID. An edge is defined by: (i) a start anchor point (StartEdge, StartPos)—i.e., the EdgeID and the corresponding base position from which the edge originates; (ii) an end anchor point (EndEdge, EndPos)—i.e., EdgeID and corresponding base position to which the edge joins; and (iii) a nucleotide sequence (Seq).

This approach of edge identification is beneficial for the backward compatibility of graph genome assemblies with a growing number of samples, as long as the definition of edges (ID and Sequence) in previous samples/versions remains intact and the version to which a group belongs is tracked. A path will still map to the same exact sequence even if the graph genome reference is switched to a newer version built on top of a previous version by the addition of more samples and edges.

While the table or matrix structure above might be useful for runtime operations, for efficient file storage, the edge sequences at varying lengths can be separately stored. Because table structures are more efficiently stored and accessed when the fields in the table have a fixed size, which would not be the case for the edge sequences. The sequences of edges belonging to the same group may be ordered by their Edge Indices, and concatenated and stored as one merged group sequence. Multiple group sequences can then be stored in, say, the FASTA format, with each sequence identified by its Group ID. The rest of the genome graph table can be stored in another file, with the column Seq replaced by SeqPos (sequence position), which marks the base position in the group sequence that corresponds to the start of the edge sequence.

A method for storing a genomic graph representing a plurality of individual genomes will now be described. Such method may be carried out by a processor. The processor first obtains and stores a linear representation of a reference genome in a data file in some sort of data storage. Next the processor receives a first genome and identifies variations in the first genome from the reference genome. Next the processor generates graph edges for each variation in the first genome from the reference genome. Then for each graph edge found in the first genome the following data are generated: an edge identifier that uniquely identifies the current edge in the genome graph; a start edge identifier that identifies the edge from which the current edge branches out; a start position that indicates the position on the start edge that serves as an anchoring point for the current edge; an end edge identifier that identifies the edge into which the current edge joins in; an end position that indicates the position on the end edge that serves as an anchoring point or the current edge; and a sequence indicating the nucleotide sequence of the current edge. The processor then stores the edge identifier, start edge identifier, start position, end edge identifier, end edge position, and sequence for each generated graph edge in the data storage. Optionally, the processor may generate for each generated graph edge a length indicating the length of the sequence and store the length in the data storage. Further, this data may be stored as a data table as described above. Also, the sequence data may be concatenated and stored separately as describe above, so that the data table has a define structure. These steps may then be repeated for each new sample that is added to the genome graph.

Defining specific paths in genome graphs will now be described. To unambiguously locate a DNA segment, in a linear reference genome, one standard way is to specify the start position and the length of the segment, where the start position is simply made up of a chromosome ID and a base coordinate or index that counts the number of bases from the beginning of the chromosome. For example, the sequence for the segment starting from “chr1:10001” (the 10001st base of Chromosome 1) with a length of 10 bases is “TAACCCTAAC”, assuming coordinates are 1-start, fully closed.

In a similar fashion, a compact way of specifying a path in a genome graph is now proposed, which may traverse one or more edges. There are three components in the specification of a path:
(i) Position—the starting point of the path, which includes a chromosome ID (ChrID), an Edge ID (EdgeID) and a base position (Pos) in a string format such as ChrID(-EdgeID):Pos, where the brackets indicate an optional component. The EdgeID may be omitted if the starting point is on the backbone sequence of the foundation group G₀. In this case, position is specified in the same way as in the linear reference.
(ii) Length—the total number of nucleotides/bases in the path.
(iii) Trail—a delimited cascade of Edge/Group IDs traversed by a path. For clarity, two different delimiter symbols may be used, e.g., “>” and “.”, respectively for branch-out and endpoint transitions. The difference between the two types of transitions is that the endpoint transition happens at the last base of the current edge, whereas a branch-out transition happens in the middle of the current edge. In general, whenever a path switches from one edge to another, the ID of the new edge should be appended to the trail. It is, however, possible to simplify and contract a trail for reducing data storage. There are different ways of contracting a trail by making different assumptions on the default priorities of the next edges. The following are some suggested rules for trail contraction:
(a) The ID of the next edge may be omitted from the trail if the path traverses the full length of the current edge and one of the following conditions holds among the edges extending from the endpoint:
   There is only one option for the next edge in the graph.
   The next edge belongs to the same group as the current edge and has the smallest Edge Index within the group among all possible options.
   The next edge belongs to the foundation Group Go (backbone sequence) and none of the plausible edges belongs to the same group as the current edge.
(b) If the branch-out and endpoint edges are differentiated with different delimiter symbols, the contraction rules in (a) may similarly be applied to the branch-out edges.
(c) Group ID may be used instead of Edge ID (with Edge Index omitted) if the next edge is the one with the smallest index within the group among the plausible edges.
(d) The contraction rules in (a), (b) and (c) may be repeatedly applied on each edge on the trail.

FIGS. 2 to 5 illustrate some path examples based on the above definitions and contraction rules and with reference to the partial genome graph defined in FIG. 1 and Table 1. In each example, the specified path is a bold line.

FIG. 2 illustrates a first example path. The path 260 in bold, solid line includes the edge G1-2 115 and otherwise follows the backbone sequence starting at position 52 of G₀ 105. The details are as follows:

Position: Chr_N:52

Length: 720 (=428+292)

Full Trail: G₁-2

Simplified Trial: G₁-2

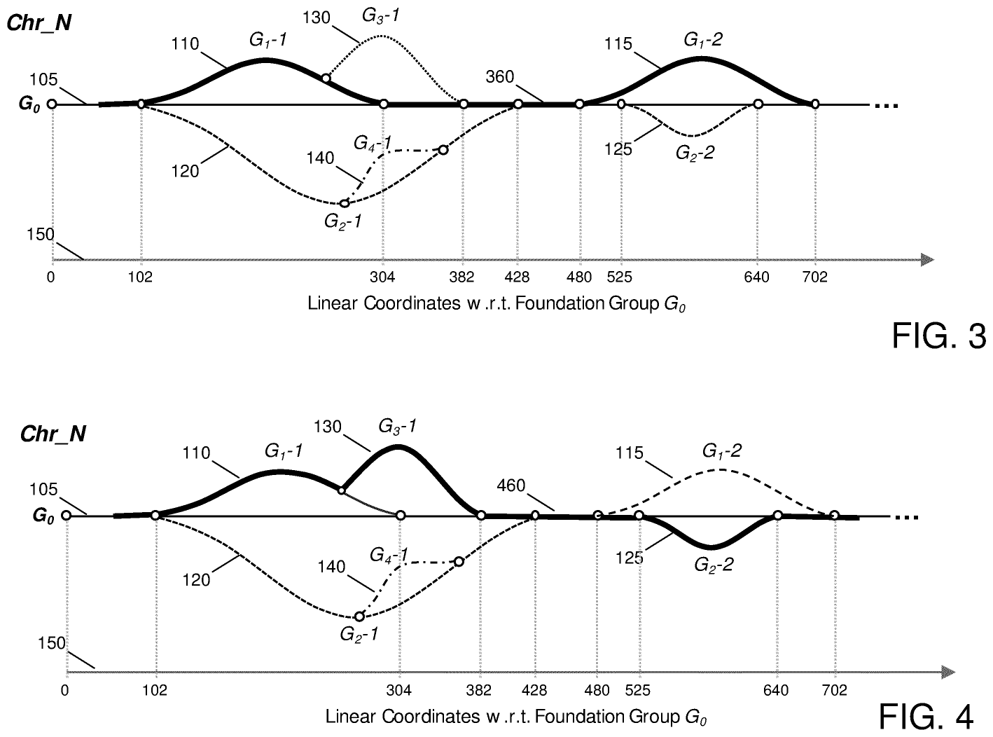

FIG. 3 illustrates a second example path. The path 360 in bold, solid line includes the edges G₁-1 110 and G₁-2 115 and otherwise follows the backbone sequence starting at position 52 of G₀ 105. The details are as follows:

Position: Chr_N:52

Length: 802 (=50+284+176+292)

Full Trail: G₁-1. G₀. G₁-2

Simplified Trial: G₁ . . . G₁

Note that for the simplified trail G₀ may be dropped because that is the only way to get between the only two segments of G₁. Also, the specific segments for G₁ may be dropped as well.

FIG. 4 illustrates a third example path. The path 460 in bold, solid line includes the edges G₁-1 110, G₃-1 130, and G₁-2 115 and otherwise follows the backbone sequence starting at position 52 of G₀ 105. The details are as follows:

Position: Chr_N:52

Length: 884 [=50+203+255+(525−382)+151+82]

With one delimiter symbol,

Full Trail: G₁-b. G₃-1. G₀. G₂-2. G₀

Simplified Trail: G₁. G₃ . . . G₂

With two delimiter symbols (“>” for branch-out, “.” for endpoint),

Full Trail: G₁-1>G₃-1. G₀. G₂-2. G₀

Simplified Trail: G₁>G₃ . . . G₂

Note that two different examples are given: one using a single delimiter symbol and another using the two different delimiter symbols. Either representation may be used.

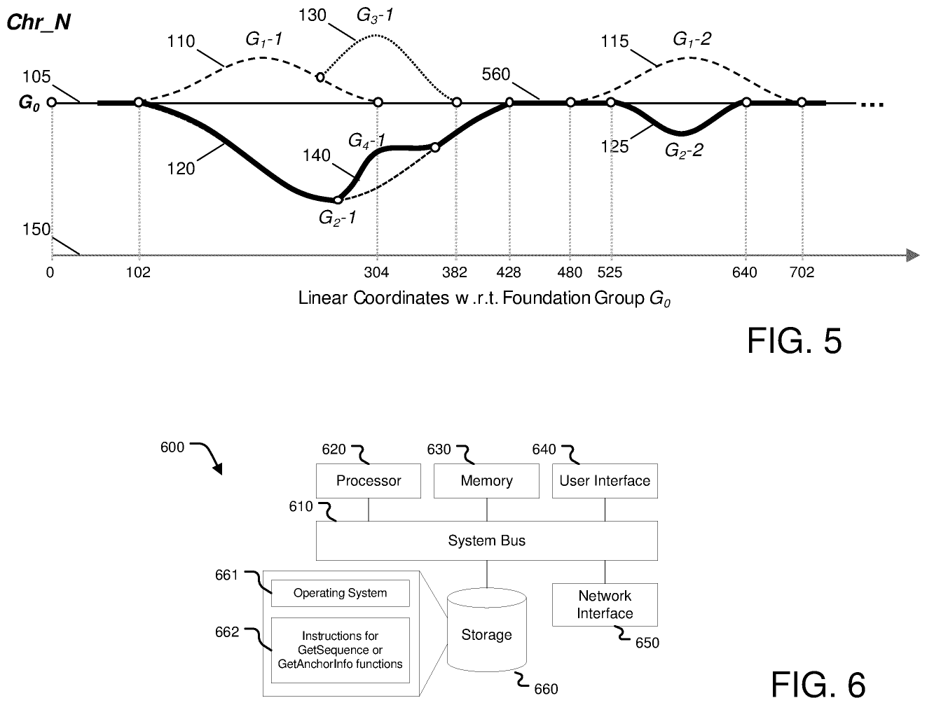

FIG. 5 illustrates a fourth example path. The path 560 in bold, solid line includes the edges G₂-1 120, G₄-1 140, and G₂-2 125 and otherwise follows the backbone sequence starting at position 52 of G₀ 105. The details are as follows:

Position: Chr_N:52

Length: 810 [=50+199+112+(420−302+1)+(525−428)+151+82)]

With one delimiter symbol,

Full Trail: G₂-1. G₄-1. G₂-1. G₀. G₂-2. G₀

Simplified Trial: G₂. G₄ . . . G₂

With two delimiter symbols (“>” for branch-out, “.” for endpoint),

Full Trail: G₂-1>G₄-1. G₂-1. G₀. G₂-2. G₀

Simplified Trial: G₂>G₄ . . . G₂

Note again that two different examples are given: one using a single delimiter symbol and another using the two different delimiter symbols.

Methods for extracting sequences and other information from genome graphs will now be described.

The following description illustrates two basic functions that operate on the proposed data structure and path specification for the extraction of sequence and location information from a genome graph. The first function is GetSequence. On successful return, the function GetSequence produces as an output a sequence that contains a string of nucleotide bases that corresponds to the path specified in the input arguments. A PathError will be returned if the specified path contains syntax errors or cannot be mapped to a unique locus in the genome graph.

The following is a suggested function signature for GetSequence.

PathError GetSequence (

| | | |
|---|---|---|
| [in] | genomeGraph | GenomeGraph, |
| [in] | startEdge | String, |
| [in] | startPos | int, |
| [in] | seqLength | int, |
| [in] | trail | String, |
| [out] | sequence | String |

) ;

The following table describes the inputs and output of the function GetSequence.

| Name | Description |
|---|---|
| genomeGraph | A GenomeGraph data structure that defines a genome graph like the |
| | example in Table 1. |
| startEdge | The edge from which the specified path begins. |
| startPos | The position on startEdge from which the specified path begins. |
| seqLength | The total length of the sequence in the specified path. |
| trail | The trail of edges traversed by the specified path. |
| sequence | The string of nucleotide bases on the specified path. |

The following pseudo-code illustrates how the GetSequence function would operate.

```
// Initialize the output sequence to an empty string.
sequence = “”;
// Initialize the current edge and position to startEdge and startPos.
currentEdge = startEdge; currentPos = startPos;
while (length(sequence) < seqLength) {
  // Get the next token in trail assuming only one delimiter symbol “.”
  // nextEdge could be empty either due to contraction, or reaching the end
  // of the trail.
  nextEdge = trail.nextToken(delimiter = “.”);
  // If nextEdge is empty, get the next edge according to the default rules.
  if (nextEdge == “”)
    nextEdge = getDefaultNextEdge(genomeGraph, currentEdge, currentPos);
  if (nextEdge == “”) {
    // If no next edge could be identified, the current edge should
    // be the last edge on the specified path.
    // Append the sequence on currentEdge from currentPos until the end.
    sequence += getEdgeSeq(genomeGraph, currentEdge, currentPos);
    break;
  } else if (isBranchOut(genomeGraph, currentEdge, nextEdge)) {
    // If it is a branch-out transition, append to the output sequence
    // only the sub-sequence on the current edge up to the branch-out
    // position. Since the path transits to the beginning of the next
    // edge, currentPos is set to 1.
    pos = getBranchPos(genomeGraph, currentEdge, nextEdge);
    sequence += getEdgeSeq(genomeGraph, currentEdge, currentPos, pos);
    currentPos = 1;
  } else {
    // If it is an endpoint transition, append to the output sequence
    // the sequence on currentEdge from currentPos until the end,
    // and set currentPos to the join-in position on the next edge.
    sequence += getEdgeSeq(genomeGraph, currentEdge, currentPos);
    currentPos = getJoinPos(genomeGraph, currentEdge, nextEdge);
  }
  currentEdge = nextEdge;
}
if (length(sequence) > seqLength)
  sequence = substring(sequence, 1, seqLength);
else
  throw PathError;
return sequence;
```

The second function is GetAnchorInfo. On successful return, the function GetAnchorInfo produces information of the anchor points on a reference edge refEdge that connect to the up- and down-stream ends of the target point (edge, edgePos) or edge (if edgePos is not specified) via the shortest paths. If the backbone sequence of the foundation group G₀ is selected to be the reference, the anchor points will be good proxies for estimating the location of the target in the linear model. A PathError will be returned if edge or edgePos does not exist in the genome graph. Note that the output variable pseudoPos (pseudo position) serves to indicate the relative position of the specified point/edge in a linear sense with respect to refEdge. The amount of deviation in a topological sense of edge from refEdge may also be measured by the number of total or branch-out only transitions as indicated by the delimiters in upAnchorTrail.

The following is a suggested function signature for GetAnchorInfo.

PathError GetAnchorInfo(

| | | |
|---|---|---|
| [in] | genomeGraph | GenomeGraph, |
| [in] | edge | String, |
| [in] | edgePos | int, |
| [in] | refEdge | String, |
| [out] | upAnchorPos | int, |
| [out] | upAnchorDist | int, |
| [out] | upAnchorTrail | String, |
| [out] | dnAnchorPos | int, |
| [out] | dnAnchorDist | int, |
| [out] | dnAnchorTrail | String, |
| [out] | pseudoPos | int |

) ;

The following table describes the inputs and outputs of the function GetAnchorInfo.

| Name | Description |
|---|---|
| genomeGraph | A GenomeGraph data structure that defines a genome graph like the |
| | example in Table 1. |
| edge | The ID of the edge whose anchor information is to be returned. |
| edgePos | The position of the point, whose anchor information is to be returned, on |
| | edge in terms of the number of bases counting from the beginning of edge. |
| | If not specified, the function will return anchor information for the two |
| | ends of edge instead. |
| refEdge | The ID of the reference edge on which the anchor points are to be found. |
| | If empty or not specified, the backbone sequence of the foundation group |
| | G₀ will be used as the reference. |
| upAnchorPos | The position of the anchor point on refEdge that connects to the upstream |
| | end of the target through the shortest path. −1 if an upstream anchor point |
| | does not exist on refEdge. |
| upAnchorDist | The distance in number of bases from the anchor point on refEdge to the |
| | upstream end of the target through the shortest path. −1 if an upstream |
| | anchor point does not exist on refEdge. |
| upAnchorTrail | The trail of edges for the shortest path from the anchor point on refEdge to |
| | the upstream end of the target. An empty string if an upstream anchor |
| | point does not exist on refEdge. |
| dnAnchorPos | The position of the anchor point on refEdge that connects to the |
| | downstream end of the target through the shortest path. −1 if a |
| | downstream anchor point does not exist on refEdge. |
| dnAnchorDist | The distance in number of bases from the anchor point on refEdge to the |
| | downstream end of the target through the shortest path. −1 if a |
| | downstream anchor point does not exist on refEdge. |
| dnAnchorTrail | The trail of edges for the shortest path from the anchor point on refEdge to |
| | the downstream end of the target. An empty string if a downstream |
| | anchor point does not exist on repEdge. |
| pseudoPos | The pseudo position of the target with respect to repEdge defined as the |
| | sum of upAnchorPos and upAnchorDist. This value gives an idea of the |
| | relative position in a linear sense. |

The GetAnchorInfo function finds the anchor points and the corresponding shortest paths by running Dijkstra's algorithm beginning from one of the endpoints of edge using the number of bases in between connecting nodes as distance until refEdge is on the spanning tree with the shortest distance among all leaf nodes, except for those that cannot be extended further.

The proposed coordinate system and data structure may be used to extend existing genomic data formats based currently on linear coordinates to support graph genome references with simple modifications. How the embodiments described herein may be applied in SAM, VCF and MPEG-G formats will now be described.

SAM File Format for Aligned Sequencing Reads

In the SAM format, each read alignment is reported on a separate line, and has 11 mandatory fields, followed by a variable number of optional fields. In particular, the mandatory fields RNAME and POS respectively specify the reference sequence name, usually chromosome number in organisms with multiple chromosomes, and left-most position where the alignment occurs. Similarly, the mandatory fields RNEXT and PNEXT are for specifying the reference sequence name and position where the mate read aligns in pair-end data.

To support genome graph references, four additional fields should be included:
(i) EDGE—ID of the specific edge to which the beginning of the read is aligned, can be left blank if it is on the backbone sequence of the foundation group G₀.
(ii) ENEXT—ID of the specific edge to which the next read of the template is primarily aligned, can be left blank if it is on the backbone sequence of the foundation group G₀.
(iii) TRAIL—the delimited sequence of edge transitions taken by the path to which the read is aligned.
(iv) TNEXT—the delimited sequence of edge transitions taken by the path to which the next read of the template is primarily aligned.
   POS and PNEXT then refer to the base positions counting from the beginning of the corresponding edges.
   Optionally, one can include the fields ANCHOR_POS and ANCHOR_PNEXT, that denote the upstream anchor position (as obtained using GetAnchorInfo) corresponding to POS and PNEXT, respectively. This would provide a means of estimating the location of the alignment in the linear model, which might be useful for sorting the file by position. Similarly, the PSEUDO_POS and PSEUDO_PNEXT returned by GetAnchorInfo can be included.

VCF File Format for Variant Calls

In the VCF format, each variant constitutes a row in a table with eight fixed, mandatory fields. The variant location is specified by the fields CHROM (chromosome ID) and POS (position on the chromosome). To support genome graph references, POS can be used to specify for each variant the position of the upstream anchor point on the backbone sequence. Two additional fields should be included:
(i) TRAIL—the sequence of edge transitions that lead to the location of the variant.
(ii) DIST—the distance in number of bases from the upstream anchor point to the variant. On the occasion when an upstream anchor point does not exist on the backbone sequence, the ID of the edge on which the variant is located should be added to the CHROM field in a format such as “ChrID-EdgeID”.

MPEG-G Standard for Compression of Genomic Data

In the MPEG-G standard, sequencing reads of the same type (i.e., alignment status, e.g., CLASS_P for perfect matching and CLASS_U for unmapped reads) are grouped into Access Units. In the Access Unit Header, a sequence_ID field is used to identify the reference sequence, e.g., a specific chromosome that an Access Unit refers to. In a Genomic Record, the field mapping_pos contains one or more mapping positions of the left-most read in the current record for multiple alignments. For each alignment in mapping_pos, if a matching segment is aligned to the same Reference Sequence, then the field delta is used to specify its position relative to the first segment. On the contrary, if a matching segment is not aligned to the same Reference Sequence, then the fields split_seq_ID and split_pos are used to specify its Reference Sequence and corresponding absolute position.

To adapt MPEG-G to support genome graphs, the following changes can be incorporated:
(i) Add a field coordinate_scheme to reference_metadata at the Dataset Group level to indicate to the decoder how the genomic coordinates should be interpreted. For example, it can be “linear” or blank for linear coordinates, or “graph v.1” for graph-based coordinate system version 1.
(ii) Use seq_ID in Genomic Record to specify an edge in a chromosome to which the beginning of the first read is mapped, e.g., in the format “ChrID(-EdgeID)”, where EdgeID can be omitted if referring to the backbone sequence of G₀. Similarly, the same format should be used for split_seq_ID to specify the beginning edges of any split segment alignments. Note that this requires the addition of a descriptor to represent the seq_ID, since in the current MPEG-G standard, all records within an access unit share the same seq_ID.
(iii) The positions in mapping_pos and split_pos should count from the beginning of the edges respectively in seq_ID and split_seq_ID.
(iv) Add a field trail in each genomic record to specify for each alignment position in mapping_pos the sequence of edge transitions to which all template segments in the same record are aligned. This also requires the addition of a new descriptor representing the trail.
(v) When the paired reads are included in the same record, the current field delta, which specifies the distance between the two reads, should be accompanied by delta_trail, which specifies the corresponding path connecting the paired reads from the 3′ end of the left read to the 5′ end of the right read.
(vi) Optionally fields corresponding to the anchor position and the pseudo position (as returned by GetAnchorInfo) for the current and split reads can be included as a means of estimating the location of the alignment in the linear model.

Although DNA sequence and genome graph are described herein, the ideas could be applied to other types of sequences such as transcriptomes and proteins.

The genome graph structure and coordinate system described herein describes a new way of storing the information for various genomes in a graph structure in a compact way that can then be used by various functions to determine and analyze various aspects of the genome graph. For example, a GetSequence function allows a sequence to be determined from the genome graph stored in a data structure using the coordinate system based upon specific input parameters. Other functions may be applied to the new graph genome coordinate system disclosed herein. Further, the coordinate system described herein may be used to extend existing genomic data formats as described above. The new coordinate system and data structure allows for a more efficient storage of multiple genomes using a genome graph and allows for efficient descriptions of new genomes (or sections thereof) as well as processing the genome graph to obtain various information. This is an improvement over the use of multiple linear reference genomes with linear coordinates. It also extends the use of genome graphs in a way that allows for the information to be efficiently stored and processed. It therefore solves the technical problem of storing and processing the genomic data for a number of different genomes. Also, solves the technical problem of storing the data for a genomic graph in a way that can be easily access and processed. These disclosed embodiments improve the operation of a computer by allowing for an ordered storage of the data for further processing, a more compact storage of genome data for a number of samples, and more efficient processing of genome data.

FIG. 6 illustrates an exemplary hardware diagram 600 for implementing a system for storing and processing genome graphs as described above. As shown, the device 600 includes a processor 620, memory 630, user interface 640, network interface 650, and storage 660 interconnected via one or more system buses 610. It will be understood that FIG. 6 constitutes, in some respects, an abstraction and that the actual organization of the components of the device 600 may be more complex than illustrated.

The processor 620 may be any hardware device capable of executing instructions stored in memory 630 or storage 660 or otherwise processing data. As such, the processor may include a microprocessor, a graphics processing unit (GPU), field programmable gate array (FPGA), application-specific integrated circuit (ASIC), any processor capable of parallel computing, or other similar devices.

The memory 630 may include various memories such as, for example L1, L2, or L3 cache or system memory. As such, the memory 630 may include static random-access memory (SRAM), dynamic RAM (DRAM), flash memory, read only memory (ROM), or other similar memory devices.

The user interface 640 may include one or more devices for enabling communication with a user. For example, the user interface 640 may include a display, a touch interface, a mouse, and/or a keyboard for receiving user commands. In some embodiments, the user interface 640 may include a command line interface or graphical user interface that may be presented to a remote terminal via the network interface 650.

The network interface 650 may include one or more devices for enabling communication with other hardware devices. For example, the network interface 650 may include a network interface card (NIC) configured to communicate according to the Ethernet protocol or other communications protocols, including wireless protocols. Additionally, the network interface 650 may implement a TCP/IP stack for communication according to the TCP/IP protocols. Various alternative or additional hardware or configurations for the network interface 650 will be apparent.

The storage 660 may include one or more machine-readable storage media such as read-only memory (ROM), random-access memory (RAM), magnetic disk storage media, optical storage media, flash-memory devices, or similar storage media. In various embodiments, the storage 660 may store instructions for execution by the processor 620 or data upon with the processor 620 may operate. For example, the storage 660 may store a base operating system 661 for controlling various basic operations of the hardware 600. The storage 661 may store instructions for carrying out the functions GetSequence or GetAnchoInfo described above. The storage 661 may also store instructions for managing the genome graph data structure.

It will be apparent that various information described as stored in the storage 660 may be additionally or alternatively stored in the memory 630. In this respect, the memory 630 may also be considered to constitute a “storage device” and the storage 660 may be considered a “memory.” Various other arrangements will be apparent. Further, the memory 630 and storage 660 may both be considered to be “non-transitory machine-readable media.” As used herein, the term “non-transitory” will be understood to exclude transitory signals but to include all forms of storage, including both volatile and non-volatile memories.

While the host device 600 is shown as including one of each described component, the various components may be duplicated in various embodiments. For example, the processor 620 may include multiple microprocessors that are configured to independently execute the methods described herein or are configured to perform steps or subroutines of the methods described herein such that the multiple processors cooperate to achieve the functionality described herein. Further, where the device 600 is implemented in a cloud computing system, the various hardware components may belong to separate physical systems. For example, the processor 620 may include a first processor in a first server and a second processor in a second server.

Any combination of specific software running on a processor to implement the embodiments of the invention, constitute a specific dedicated machine.

As used herein, the term “non-transitory machine-readable storage medium” will be understood to exclude a transitory propagation signal but to include all forms of volatile and non-volatile memory.

Although the various exemplary embodiments have been described in detail with particular reference to certain exemplary aspects thereof, it should be understood that the invention is capable of other embodiments and its details are capable of modifications in various obvious respects. As is readily apparent to those skilled in the art, variations and modifications can be affected while remaining within the spirit and scope of the invention. Accordingly, the foregoing disclosure, description, and figures are for illustrative purposes only and do not in any way limit the invention, which is defined only by the claims.

What is claimed is:

1. A method for storing, by a processor, a genomic graph representing a plurality of individual genomes, comprising:
storing a linear representation of a reference genome in a data storage;
receiving a first genome;
identifying variations in the first genome from the reference genome;
generating graph edges for each variation in the first genome from the reference genome;
generating for each generated graph edge:
an edge identifier that uniquely identifies the current edge in the genome graph;
a start edge identifier that identifies the edge from which the current edge branches out;
a start position that indicates the position on the start edge that serves as an anchoring point for the current edge;
an end edge identifier that identifies the edge into which the current edge joins in;
an end position that indicates the position on the end edge that serves as an anchoring point for the current edge; and
a sequence indicating the nucleotide sequence of the current edge; and
storing the edge identifier, start edge identifier, start position, end edge identifier, end edge position, and sequence for each generated graph edge in the data storage.

2. The method claim 1, further comprising:
generating for each generated graph edge a length indicating the length of the sequence; and
storing the length in the data storage.

3. The method claim 2, wherein the edge identifier, start edge identifier, start position, end edge identifier, end edge position, sequence, and length for each generated graph edge are stored as a row in a data table.

4. The method claim 1, wherein the edge identifier, start edge identifier, start position, end edge identifier, end edge position, and sequence for each generated graph edge are stored as a row in a data table.

5. The method claim 4, further comprising concatenating the sequences for each of the generated edges and storing them in a sequence data structure separate from the data table.

6. The method claim 1, further comprising specifying a path in the genome graph, wherein the path is defined by:
a position indicating the starting point of the path including a chromosome identifier; the edge identifier, and a base position;
a path length indicating the total number of nucleotides in the path; and
a trail including a cascade of edge identifiers traversed by the path using delimiters.

7. The method claim 6, wherein the delimiters include a branch-out delimiter and an endpoint delimiter.

8. The method claim 6, further comprising simplifying the trail by removing edge identifiers from the trail based upon assumptions of the default priorities of the next edges.

9. The method claim 1, wherein the edge identifier includes a group identifier and an edge index, wherein the group identifier identifies a group of related edges and the edge index uniquely identifies an edge within a group.

10. The method claim 9, wherein the group identifier identifies a group of edges from a same origin, such as an individual sample.

11. The method claim 1, further comprising extending a sequencing read alignment file (SAM) by adding the additional data fields:
the edge identifier of the specific edge to which the beginning of the read is aligned;
an ENEXT identifier of the specific edge to which the next read of the template is primarily aligned;
a trail indicating the delimited sequence of edge transitions taken by the path to which the read is aligned; and
TNEXT indicating the delimited sequence of edge transitions taken by the path to which the next read of the template is primarily aligned.

12. The method claim 1, further comprising extending a variant call file (VCF) by adding the additional data fields:
a trail indicating sequence of edge transitions that lead to the location of the variant; and
a distance indicating the distance in the number of bases from the upstream anchor point to the variant.

13. The method claim 1, further comprising extending a MPEG-G file by
adding the additional data fields:
a coordinate_scheme field that indicates to a decoder how the genomic coordinates should be interpreted; and
a trail field in each genomic record to indicate for each alignment position in mapping_pos the sequence of edge transitions to which all template segments in the same record are aligned;
using the MPEG-G file data fields as follows:
the seq_ID indicates an edge in a chromosome to which the beginning of the first read is mapped;
the split_seq_ID indicates the beginning edges of any split segment alignments; and
the positions mapping_pos and split_pos count from the beginning of the edges respectively in seq_ID and split_seq_ID.

14. The method claim 1, further comprising determining anchor points on a specified edge by extending a MPEG-G file by running Dijkstra's algorithm beginning from one of the endpoints of specified edge using a number of bases in between connecting nodes as distance until the specified edge is on a spanning tree with a shortest distance among all leaf nodes, except for those that cannot be extended further.

15. The method claim 1, further comprising determining the sequence along a path in the genome sequence further comprising:
traversing a trail defining the path, wherein the trail includes a cascade of edge identifiers traversed by the path; and
concatenating the sequences of each of the edges identified along the path.

16. A non-transitory machine-readable storage medium encoded with instructions for storing, by a processor, a genomic graph representing a plurality of individual genomes, the non-transitory machine-readable storage medium comprising:
instructions for storing a linear representation of a reference genome in a data storage;
instructions for receiving a first genome;
instructions for instructions for identifying variations in the first genome from the reference genome;
instructions for generating graph edges for each variation in the first genome from the reference genome;
instructions for generating for each generated graph edge:
an edge identifier that uniquely identifies the current edge in the genome graph;
a start edge identifier that identifies the edge from which the current edge branches out;
a start position that indicates the position on the start edge that serves as an anchoring point for the current edge;
an end edge identifier that identifies the edge into which the current edge joins in;
an end position that indicates the position on the end edge that serves as an anchoring point for the current edge; and
a sequence indicating the nucleotide sequence of the current edge; and
instructions for storing the edge identifier, start edge identifier, start position, end edge identifier, end edge position, and sequence for each generated graph edge in the data storage.

17. The non-transitory machine-readable storage medium claim 16, further comprising:
instructions for generating for each generated graph edge a length indicating the length of the sequence; and
instructions for storing the length in the data storage.

18. The non-transitory machine-readable storage medium claim 17, wherein the edge identifier, start edge identifier, start position, end edge identifier, end edge position, sequence, and length for each generated graph edge are stored as a row in a data table.

19. The non-transitory machine-readable storage medium claim 16, wherein the edge identifier, start edge identifier, start position, end edge identifier, end edge position, and sequence for each generated graph edge are stored as a row in a data table.

20. The non-transitory machine-readable storage medium claim 19, further comprising instructions for concatenating the sequences for each of the generated edges and storing them in a sequence data structure separate from the data table.

21. The non-transitory machine-readable storage medium claim 16, further comprising instructions for specifying a path in the genome graph, wherein the path is defined by:
a position indicating the starting point of the path including a chromosome identifier; the edge identifier, and a base position;
a path length indicating the total number of nucleotides in the path; and
a trail including a cascade of edge identifiers traversed by the path using delimiters.

22. The non-transitory machine-readable storage medium claim 21, wherein the delimiters include a branch-out delimiter and an endpoint delimiter.

23. The non-transitory machine-readable storage medium claim 21, further comprising instructions for simplifying the trail by removing edge identifiers from the trail based upon assumptions of the default priorities of the next edges.

24. The non-transitory machine-readable storage medium claim 16, wherein the edge identifier includes a group identifier and an edge index, wherein the group identifier identifies a group of related edges and the edge index uniquely identifies an edge within a group.

25. The non-transitory machine-readable storage medium claim 24, wherein the group identifier identifies a group of edges from a same origin, such as an individual sample.

26. The non-transitory machine-readable storage medium claim 16, further comprising instructions for extending a sequencing read alignment file (SAM) by adding the additional data fields:
the edge identifier of the specific edge to which the beginning of the read is aligned;
an ENEXT identifier of the specific edge to which the next read of the template is primarily aligned;
a trail indicating the delimited sequence of edge transitions taken by the path to which the read is aligned; and
TNEXT indicating the delimited sequence of edge transitions taken by the path to which the next read of the template is primarily aligned.

27. The non-transitory machine-readable storage medium claim 16, further comprising instructions for extending a variant call file (VCF) by adding the additional data fields:
a trail indicating sequence of edge transitions that lead to the location of the variant; and
a distance indicating the distance in the number of bases from the upstream anchor point to the variant.

28. The non-transitory machine-readable storage medium claim 16, further comprising extending a MPEG-G file by
adding the additional data fields:
a coordinate_scheme field that indicates to a decoder how the genomic coordinates should be interpreted; and
a trail field in each genomic record to indicate for each alignment position in mapping_pos the sequence of edge transitions to which all template segments in the same record are aligned;
using the MPEG-G file data fields as follows:
the seq_ID indicates an edge in a chromosome to which the beginning of the first read is mapped;
the split_seq_ID indicates the beginning edges of any split segment alignments; and
the positions mapping_pos and split_pos count from the beginning of the edges respectively in seq_ID and split_seq_ID.

29. The non-transitory machine-readable storage medium claim 16, further comprising instructions for determining anchor points on a specified edge by extending a MPEG-G file by running Dijkstra's algorithm beginning from one of the endpoints of specified edge using a number of bases in between connecting nodes as distance until the specified edge is on a spanning tree with a shortest distance among all leaf nodes, except for those that cannot be extended further.

30. The non-transitory machine-readable storage medium claim 16, further comprising instructions for determining the sequence along a path in the genome sequence further comprising:
traversing a trail defining the path, wherein the trail includes a cascade of edge identifiers traversed by the path; and
concatenating the sequences of each of the edges identified along the path.
